(19) United States
(12) Patent Application Publication
(10) Pub. No.: US 2016/0262931 A1
(43) Pub. Date: Sep. 15, 2016

(54) MICROSURGICAL FORCEPS WITH WIDE JAWS

(71) Applicant: Suven Bhattacharjee, Kolkata (IN)

(72) Inventor: Suven Bhattacharjee, Kolkata (IN)

(21) Appl. No.: 14/686,991

(22) Filed: Apr. 15, 2015

(30) Foreign Application Priority Data
Mar. 9, 2015 (IN) 245/KOL/2015

(51) Int. Cl. A61F 9/007; A61B 17/29; A61F 2/16

(52) U.S. Cl. CPC A61F 9/007; A61F 2/1664; A61B 17/29; A61B 2017/2926; A61B 2017/294

(57) ABSTRACT

A forceps for microsurgery having a tubular shaft with a coaxially placed central rod, an upper shank with a proximate end attached to the tubular shaft and a distal end with an upper jaw attached thereto, and a lower shank with a proximate end attached to the distal central rod and a distal end with a lower jaw attached thereto. The upper and lower jaws both have diameters greater than the outer diameter of the tubular shaft, and may be offset from the upper and lower shanks, respectively, so as to facilitate microsurgical maneuvers such as lifting and grasping thin objects or tissue edges positioned on the surface of an iris or lens during an ophthalmological surgical procedure.

### CROSS REFERENCE TO RELATED APPLICATION

This application claims the benefit of Indian Provisional Patent Application No. 245/KOL/2015, filed Mar. 9, 2015, the disclosure of which is incorporated by reference herein in its entirety.

### BACKGROUND OF THE INVENTION

The present invention is in the field of minimally invasive surgery. More particularly the invention is an improvement in microsurgical forceps which are tubular shaft instruments with an actuating mechanism at one end and end-effector gripper jaws at the other end.

Minimally invasive surgery in the form of ocular surgery, endoscopic surgery, laparoscopic surgery or any other form involves surgical procedures through small incisions or narrow openings. Small incisions cause less pain, allow quick healing and early recovery. Certain body spaces and cavities have narrow openings as the only access. Tubular shaft instruments allow access to the inside of a body cavity, narrow space or closed space to perform different surgical steps with least trauma. Such surgical steps are performed under direct visualization, through a surgical microscope or endoscopic visualization. Microsurgery is surgery performed on very small structures with specialized instruments under a microscope. Such specialized instruments are often referred to as microsurgical instruments. Microsurgical forceps is one such instrument.

While squeeze-handle microsurgical forceps have different designs, the basic mechanism of closure of the jaws remains the same. The rigid tubular shaft comprises a hollow outer tube, which within its lumen coaxially supports a rod. The grasping forceps jaws are connected to the distal end of the rod by shanks. In the unrestrained state the jaws remain open and part of the shanks protrude from the distal end of the tubular shaft. The two jaws and the two shanks are reciprocally biased and move away from each other when unrestrained. The actuating mechanism advances the distal tubular shaft over the shanks to approximate the shanks and jaws towards each other causing closure of the jaws. Releasing the actuating mechanism causes an opposite action and opens the jaws.

In certain situations in cataract surgery, it is necessary to introduce a microsurgical forceps into the anterior chamber of the eye through a small incision and hold and manipulate different tissues or objects at different parts of the anterior chamber in a single entry. This in turn means that these objects or tissues would be positioned on either side of the jaws of the forceps. Such a need typically arises with the use of the pupil-expansion device disclosed in U.S. patent application Ser. No. 14/379,684, International Patent Application No. PCT/IN2013/000457 and Indian Patent Application No. 225/KOL/2013, all to Bhattacharjee. The flanges of the pupil expansion device have to be drawn centrally from different parts of the peripheral anterior chamber and tucked under the pupil margin. It is also desired that this device be deployed using a forceps through a single small incision in the cornea in a single entry. The anterior chamber of the eye is a shallow dome with the average central depth being 3.0 mm and the depth being very shallow towards the periphery. The anterior chamber is lined by the delicate endothelium on one side and the iris and lens capsule on the other. These factors permit only translational motion of instruments in the anterior chamber with very little scope for rotation. The difficulty with available microsurgical forceps is that the jaws are so designed that they cannot hold objects or tissue edges positioned at the sides (i.e., edges) of the jaws. This difficulty increases if the object or tissue edge is oriented substantially parallel to the long axis of the jaws and the shaft.

A tubular-shaft forceps (rigid or flexible shaft) or microsurgical forceps is designed such that the tubular shaft snugly fits in the incision (corneal or scleral) or cannula (trocar-cannula) or working channel (endoscope) through which it passes. The outer diameter (OD) of the tubular shaft is essentially slightly less than that of the incision, cannula or channel. To permit free passage through the incision (corneal or scleral) or cannula (trocar-cannula) or working channel (endoscope), the largest diameter of the forceps jaws, when in a closed position (OD_{J}), is less than or equal to that of the outer diameter (OD) of the tubular shaft (OD_{S}). This precludes the jaws from gripping an object or tissue edge positioned alongside the jaws and oriented substantially parallel to the long axis of the jaws and the shaft. This difficulty is pronounced when the incision location, size or number is unfavorable or when the space for movement of the forceps is restricted.

However, this difficulty may be overcome if the jaws are wider than the shaft, like a head on a neck. If these wider than shaft jaws are also made thin, they can be passed through the same size incision (corneal or scleral) or cannula (trocar-cannula) or working channel (endoscope), provided the passage is flexible allowing its cross section to change between a circular, elliptical and slit shapes. The maximum outer diameter of the jaws (MaxOD_{J}) for a given combined thickness of the closed jaws (T_{J}) and outer diameter of shaft (OD_{S}) and circumference of shaft (Circumₛ) for such a flexible slit passage such as a corneal or scleral incision (CSI) can be calculated by the formula below.

CSI=½ Circumₛ and also, CSI=MaxOD_{J}+T_{J}

Circumₛ=π(OD_{S})

The width of corneal or scleral slit incision (CSI) would be=½{π(OD_{S})}

Hence, MaxOD_{J}+T_{J}=½ {π(OD_{S})},

Therefore, MaxOD_{J}=½{π(OD_{S})}−T_{J}

Typically, for a 23 gauge forceps where the OD of the shaft (OD_{S}) is 0.64 mm, if the combined thickness of the closed jaws (T_{J}) is 0.2 mm then maximum outer diameter of the jaws (MaxOD_{J}) is calculated as follows:

MaxOD_{J}=½{π(OD_{S})}−T_{J}=½{3.14(0.64)}−0.2=0.8 mm

For a corneal or scleral incision the outer diameter of the jaws may be a little larger than the maximum allowed by the formula (MaxOD_{J}) because the corneal or sclera incision can be stretched to some extent. The shaft would still snugly fit in the incision with no leakage. Further, for corneal incisions the jaws can be significantly larger than the shaft because the high molecular weight viscoelastic used can maintain the depth of the anterior chamber even with small amounts of leakage along sides of the shaft when the shaft does not fit snugly.

Disc-like round jaws would provide the best advantage in terms of safety, all round gripping, and no snagging during entry and exit through the slit corneal incision. If the jaws are like rings, the central hole would allow visibility of the object being grasped and the structures at deeper planes.

The AE-4907 microsurgical forceps from ASICO LLC (Illinois, USA) has ring jaws, and is described at the website and in the brochure BR419-10 at: http://www.asico.com/procedures/intraocular-complications-management-surgery/chee-subluxated-iol-grasping-forceps-21g.html#.VO9XHPmUeSo (accessed 18 Feb. 2015). The AE-4907 is a squeeze handle microsurgery forceps with a curved shaft with its concavity anteriorly. When the actuating mechanism is activated in a pincer grip, it produces appositional movement or closure of the jaws in a direction substantially perpendicular to the actuators and in a plane perpendicular to the iris plane.

Though the AE-4907 microsurgical forceps has ring jaws and the shank is narrow where it joins the ring, it cannot be used to hold objects or tissues positioned on either side of the jaws. This is because the maximum diameter of the ring jaws is less than that of the broader part of the shank and the tubular shaft. Moreover, in the closed position the tubular shaft stops fairly short of the ring effectively rendering the jaws very long. These long jaws are useful in holding a thicker (1.0 mm or more) subluxated intraocular lens for which it is meant. They are not useful in getting a firm grip on thinner (about 0.1 mm) and narrower objects.

Another difficulty with the AE-4907 microsurgical forceps is that the edges of the jaws are thick in order to get a firm grip on a subluxated intraocular lens (IOL). The jaws are also in the same level as the shank. It is difficult to pass the thick edge of the lower jaw under thin objects or tissue edges positioned on the surface of the iris and to get the platform of the jaw under the object to be grasped. Instead, in the effort to do so the thin objects or tissue edges get displaced.

A microsurgical forceps that has thin edge ring jaws which are wider than the tubular shaft, and which are posteriorly set on the shank as well as posteriorly offset in relation to the shank, is more effective in lifting and grasping thin objects or tissue edges positioned on the surface of the iris or lens. The lower jaw passes under the thin objects or tissue edges like a cement trowel.

### OBJECTS OF THE INVENTION

The principal object of the present invention is to provide a microsurgical forceps with jaws that can grip an object or tissue edge positioned alongside the jaws and oriented substantially parallel to the long axis of the jaws and the shaft. Another object of the invention is to provide a microsurgical forceps with jaws that can grip objects or tissue edges positioned on either side of the jaws without having to rotate the forceps inside the eye or without having to remove it from the eye and reinsert it. Another object of the invention is to provide a microsurgical forceps with jaws that can grip objects or tissue edges and move them in a translational motion substantially parallel to the iris plane. Another object of the invention is to provide a microsurgical forceps with jaws which allow visualization through the jaws, of the object being held and the deeper structures. Another object of the invention is to provide a microsurgical forceps for insertion, deployment and removal of the aforementioned Bhattacharjee pupil-expansion devices through a very small corneal incision. Another object of the invention is to provide a microsurgical forceps for insertion of a device, tissue or object into the eye through a very small corneal incision. Another object of the invention is to provide a microsurgical forceps for deployment of a device, tissue or object through a very small corneal incision. Another object of the invention is to provide a microsurgical forceps for removal of a device, tissue or object from the eye through a very small corneal incision. Another object of the invention is to provide a microsurgical forceps for manipulation of devices, objects or tissues in the anterior chamber of the eye. Another object of the present invention is to provide a microsurgical forceps for the aforesaid purposes, which is easily held in a pincer grip while the operators hand is resting at the plane of the incision or a higher plane. Such instruments can be used in a variety of surgeries, such as but not limited to cataract, vitreo-retinal, iris, anterior chamber, laparoscopic and endoscopic surgery.

### SUMMARY OF THE INVENTION

The present invention is a microsurgical forceps with a rigid tubular shaft, with an actuating handle on one end and end effector gripper jaws at the other end. The novelty of the instrument is that the gripper jaws are wider than the tubular shaft in the closed position of the jaws. This allows grasping of objects and tissues positioned along either side of the jaws unlike any other microsurgical forceps. The increase in width of the jaws may be compensated for by a reduction in their thickness such that they are able to pass through the same size incision as would snugly accommodate the shaft. Further, these thin jaws are set posteriorly on each shank as well as posteriorly offset in relation to the shank so as to allow the lower jaw to scoop up the object to be held like a cement trowel. The specially designed large gripper jaws allow easy atraumatic grasping of a large part of the object or tissue. They also allow sequential manipulation of multiple objects and tissues in the anterior chamber of the eye in a single entry through a single small incision in the cornea while simultaneously providing visualization of these objects or tissues and deeper structures. None of the available microsurgical forceps can sequentially hold objects or tissues in the anterior chamber of the eye positioned on either side the jaws and oriented substantially parallel to the long axis of the jaws and the shaft, in a single entry through a single small incision in the cornea. Available micro-surgical forceps require multiple entries or multiple incisions or swapping of instruments or dangerous movements within the anterior chamber of the eye to serve this purpose.

Each of the two jaws of the microsurgical forceps of the present invention is a thin plate with a large central hole and resembles a washer. The horizontal outer diameter of the jaws is larger than the diameter of the tubular shaft. Each jaw also has a central hole bound by the inner diameter. Each jaw has a ring shaped platform opposite the platform of the other jaw. The platform is bound by an outer diameter and an inner diameter. The side of each jaw has a thickness. The jaws are attached to shanks which continue as the coaxial rod within the tubular shaft. When the forceps is held in a pincer grip, the ring shaped jaws open and close in a plane substantially perpendicular to the iris plane and the direction of movement of the actuating levers. The large central hole allows visualization of the object being grasped and also allows view of the structures lying posterior to the jaws. The combined vertical thickness of the closed jaws is less than the combined vertical thickness of the shanks in the closed position of the jaws. The jaws are vertically thinner than the shanks and are attached to the posterior part of the vertical thickness of the shanks. The shanks may or may not have a gap between them in the closed position of the jaws. The vertical thickness of the shanks may be equal or unequal in different embodiments. The shanks have a beveled face where they are attached to the jaw. This allows smooth movement of the instrument through a small incision. The jaws can grasp objects in front or ahead of the rounded tips like any other forceps. The horizontal diameter of the jaws being larger than that of the tubular shaft, they allow grasping of objects or tissues on either side too. The ring shape of the jaws allows easy alignment with the object or tissue to be held, irrespective of its orientation. In the preferred embodiment fine grooves on both the opposing platforms of the jaws prevent the held object from slipping. Alternate embodiments may have sandblasted or diamond dusted platform surfaces. In the preferred embodiment both of the jaws are posteriorly offset in relation to the shanks such that at least a part of the thickness is lower than the posterior boundary of the shank creating a step effect. The sides (i.e., edges) of the jaws are rounded to eliminate sharp edges reducing the risk of trauma to tissues and facilitate scooping of the object to be held. The rounded edges, low setting and offset of the jaw facilitate sliding of the lower jaw beneath the object or tissues like a cement trowel so as to place the platform of the jaw under it. This typically allows the lower jaw to pass easily under a flange of a pupil expansion device for grasping. The offset also helps in tucking the held flange under the pupil margin by carrying it below the plane of the pupil margin.

The two shanks may be connected to the central rod differently in different embodiments of the invention. In one embodiment the two shanks may have a very narrow gap between them when the jaws are closed. In this embodiment the shanks form a very narrow V at the central rod. In another embodiment the two shanks may be thinner and form a wider gap between them when the jaws are closed. In this embodiment the shanks form a U at the central rod.

While in the preferred embodiment the two shanks are of unequal thickness, in another embodiment the shanks are of equal thickness. While in the preferred embodiment the jaws are posteriorly offset in relation to the shank, in another embodiment the lower boundary of the lower jaw is in line with the lower boundary of the lower shank and not offset. Such posteriorly offset or low set jaws are better suited for grasping objects or tissues in the deeper planes of the anterior chamber closer to the surface of the iris or lens. In another embodiment the jaws are at the mid position in relation to the cross section of the tubular shaft. Such mid-position jaws are better suited for grasping objects or tissues in the mid-planes of the anterior chamber. In another embodiment the jaws are anteriorly offset in relation to the shanks. Such anteriorly offset or high set jaws are better suited for grasping objects or tissues in the anterior planes of the anterior chamber closer to the cornea and endothelium.

In the preferred embodiment of the invention the two jaws are symmetrical (i.e., have the same width/diameter). In another embodiment the jaws are asymmetrical with the platform of the lower jaw having a greater width than that of the upper jaw. Another embodiment of the invention has disc-shaped jaws with no fenestration. Another embodiment has a disc-shaped lower jaw with no hole and a ring-shaped upper jaw with a hole.

The present invention includes micro surgical forceps used in surgery such as but not limited to cataract, vitreo-retinal, iris, anterior chamber, laparoscopic and endoscopic surgery. All such instruments have jaws which are wider than the shaft when the jaws are in the closed position.

### BRIEF DESCRIPTION OF THE DRAWINGS

FIG. 1 is a side view of the AE-4907 microsurgical forceps which is a prior art device.

FIG. 2 is an enlarged side view of the end of the forceps of FIG. 1 with open jaws.

FIG. 3 is an enlarged diagrammatic illustration of a top view of the end of the forceps of FIG. 1 with the closed jaws advanced through a corneal incision to grasp objects in the anterior chamber of the eye.

FIG. 4 is an enlarged diagrammatic illustration of a top view of the distal end of a preferred embodiment of the microsurgical forceps of the present invention advanced through a corneal incision and grasping an object in the anterior chamber of the eye between the jaws of the forceps, with another object also being in the anterior chamber.

FIG. 5 is an enlarged diagrammatic side view of open jaws, shanks and distal end of the tubular shaft of a preferred embodiment of the microsurgical forceps of the present invention.

FIG. 6 is an enlarged diagrammatic side view of closed jaws and distal end of the tubular shaft of the microsurgical forceps of FIG. 5.

FIG. 7 is an enlarged diagrammatic view of a longitudinal section through the middle of the posteriorly offset closed jaws, unequally thick shanks and distal end of the tubular shaft of the microsurgical forceps of FIG. 5.

FIG. 8 is an enlarged diagrammatic view of a longitudinal section through the middle of the posteriorly offset closed jaws, equally thick shanks and distal end of the tubular shaft of an embodiment of the microsurgical forceps of the present invention.

FIG. 9 is an enlarged diagrammatic side view of the closed jaws and distal tubular shaft of an embodiment of the microsurgical forceps of the present invention with the lower boundary of the lower jaw being level with the lower boundary of the lower shank.

FIG. 10 is an enlarged diagrammatic view of a longitudinal section through the middle of the closed jaws and distal shaft of an embodiment of the microsurgical forceps of the present invention with closed jaws in mid position in relation to the distal end of the tubular shaft with thin shanks connected to a central rod.

FIG. 11 is an enlarged diagrammatic view of a longitudinal section through the middle of the anteriorly offset closed jaws, equally thick shanks and distal end of the tubular shaft of an embodiment of the microsurgical forceps of the present invention.

### DETAILED DESCRIPTION OF DRAWINGS

The relevant features of existing devices and the novelty of the present invention, is illustrated in the accompanying drawings, throughout which, like reference numerals indicate corresponding parts in the various figures.

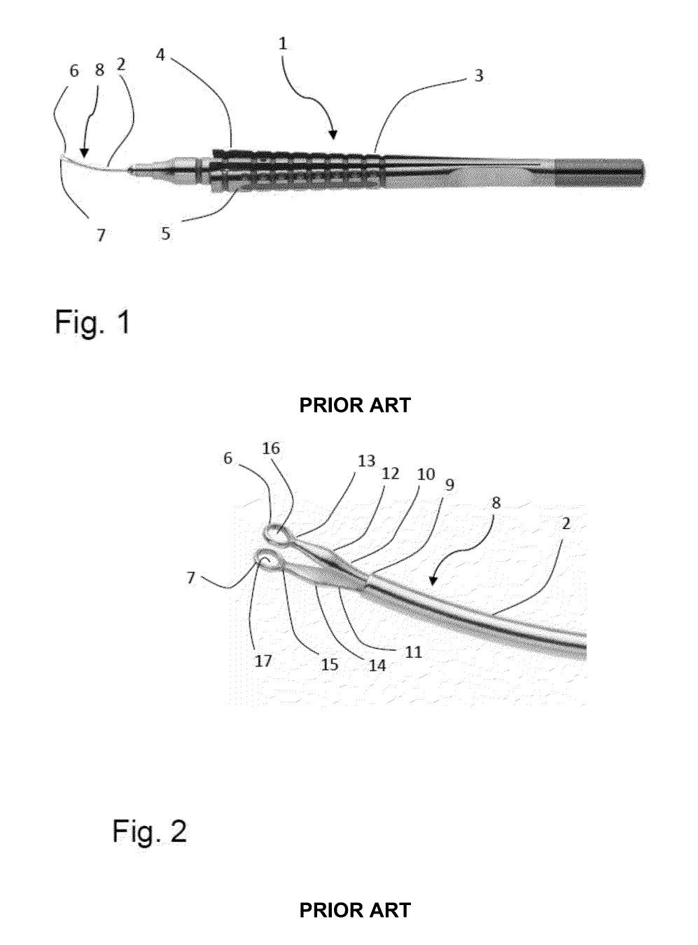

FIG. 1 shows a side view of the AE-4907 microsurgical forceps 1 which is a prior art device showing a tubular shaft 2 with a handle 3 and actuators 4 and 5 at one end and ring jaws 6 and 7 at the other end 8. The tubular shaft 2 is curved with its concavity anteriorly. The upper jaw 6 is placed anteriorly and the lower jaw 7 is placed posteriorly. Squeezing the actuators 4 and 5 in a pincer grip causes closure of the jaws 6 and 7 in a direction substantially perpendicular to iris plane and the movement of the actuators.

FIG. 2 is an enlarged side view of end 8 of the forceps 1 of FIG. 1 showing tubular shaft 2 and jaws 6 and 7 in the open position. The tubular shaft 2 has a distal end 9 towards the jaws 6 and 7. In the open position of the jaws, anteriorly placed upper shank 10 and posteriorly placed lower shank 11 emerge from the distal end 9 of the tubular shaft 2. The upper shank 10 has a broad part 12 leading to a narrow neck 13 where it joins the anteriorly placed ring shaped upper jaw 6. The lower shank 11 has a broad part 14 leading to a narrow neck 15 where it joins the posteriorly-placed ring-shaped lower jaw 7. The ring-shaped upper jaw 6 has a central hole 16 and the ring shaped lower jaw 7 has a central hole 17. Squeezing the actuator mechanism slides the distal end 9 of the tubular shaft 2 over the shanks 10 and 11 towards the ring jaws 6 and 7 causing their approximation. In the fully closed position of the jaws, the distal end 9 of the tubular shaft 2 advances up to the broad part 12 of the upper shank 10 and the broad part 14 of the lower shank 11.

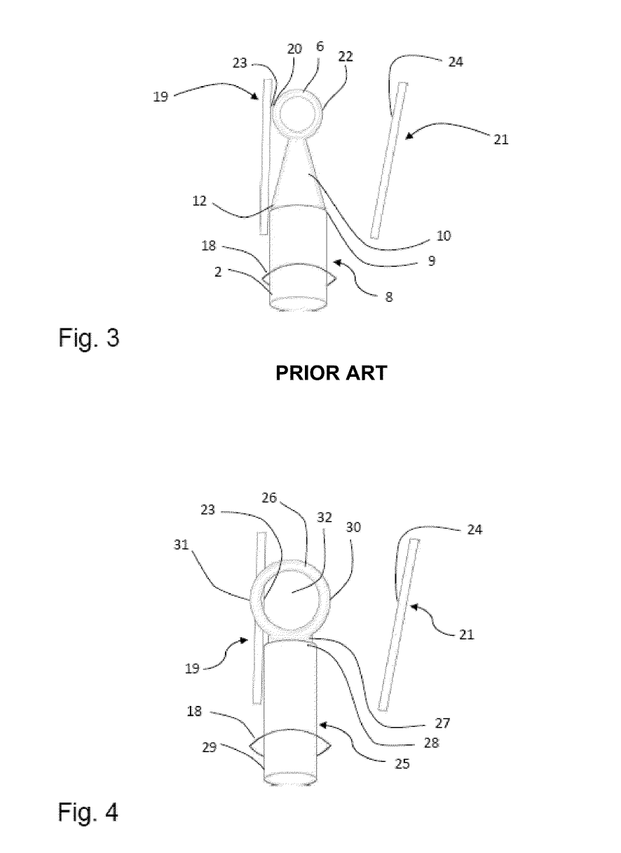

FIG. 3 is an enlarged diagrammatic illustration of a top view of end 8 of forceps 1 of FIG. 1 with jaws closed and only upper ring jaw 6, upper shank 10 and front of distal end 9 of the tubular shaft 2 being visible. The forceps is advanced with jaws closed through the corneal incision 18 to grasp sequentially an object 19 positioned alongside the left edge 20 of upper ring jaw 6 and an object 21 positioned obliquely about one and half jaw diameters away and to the right of the right edge 22 of the jaw 6. Corneal incision 18 is the only access to the objects 19 and 21 positioned in the anterior chamber of the eye. The object 19 needs to be grasped at point 23 on its right edge between the left sides of the platforms of the ring jaws and manipulated in a translational motion. Soon after the object 19 is released, without exiting the anterior chamber of the eye, object 21 needs to be grasped at point 24 on its left edge between the right sides of the platforms of the ring jaws and manipulated in a translational motion. The diameter of the ring jaw 6 is equal to the broadest part 12 of the upper shank 10 and less than the outer diameter of the tubular shaft 2. Because object 19 is positioned substantially parallel and close to shaft 2 and the upper ring jaw 6 has a small diameter, the left edge 20 of jaw 6 is unable to pass over point 23 on the right edge of object 19 in order to grasp it. As the forceps is moved obliquely to grasp point 24 on the left edge of object 21, the object 21 now becomes positioned substantially parallel and close to shaft 2 and because the upper ring jaw 6 has a small diameter, the right edge 22 of jaw 6 is unable to pass over the point 24 on the left edge of object 21 in order to grasp it. The part of the shank 10 distal to broad part 12 effectively becomes a part of the jaw because it remains outside the distal part 9 of the shaft when the ring jaws are closed.

FIG. 4 is an enlarged diagrammatic illustration of a top view of end 25 of a preferred embodiment of the forceps of the present invention with jaws closed and only upper ring jaw 26, small part of upper shank 27, front of distal end 28 of the tubular shaft 29 being visible. Another object 21 is positioned obliquely about one and half jaw diameters away and to the right of the right edge 30 of the jaw 26. Corneal incision 18 is the only access to the objects 19 and 21 positioned in the anterior chamber of the eye. The forceps has been advanced through the corneal incision 18 and has grasped object 19 positioned substantially parallel and close to the tubular shaft 28, between the left sides of the platforms of the jaws. The diameter of the upper ring jaw 26 is greater than the upper shank 27 and the outer diameter of the tubular shaft 29. Because the upper ring jaw 26 has a relatively large diameter, the left edge 31 of upper jaw 26 is able to pass over point 23 on the right edge of object 19 in order to grasp it. The large hole 32 allows visualization of point 23 on the right edge of object 19. Object 19 needs to be grasped at point 23 between the left sides of the platforms of the ring jaws and manipulated in a translational motion. Soon after the object 19 is released, without exiting the anterior chamber of the eye, object 21 needs to be grasped at point 24 on its left edge between the right sides of the platforms of the ring jaws and manipulated in a translational motion. As the forceps is moved obliquely to grasp point 24 on the left edge of object 21, the object 21 now becomes positioned substantially parallel and close to shaft 29 and because the upper ring jaw 26 has a relatively large diameter, the right edge 30 of upper jaw 26 is able to pass over the point 24 on the left edge of object 21 in order to grasp it. Only a small part of the shank 27 remains outside the distal part 28 of the shaft when the ring jaws are closed.

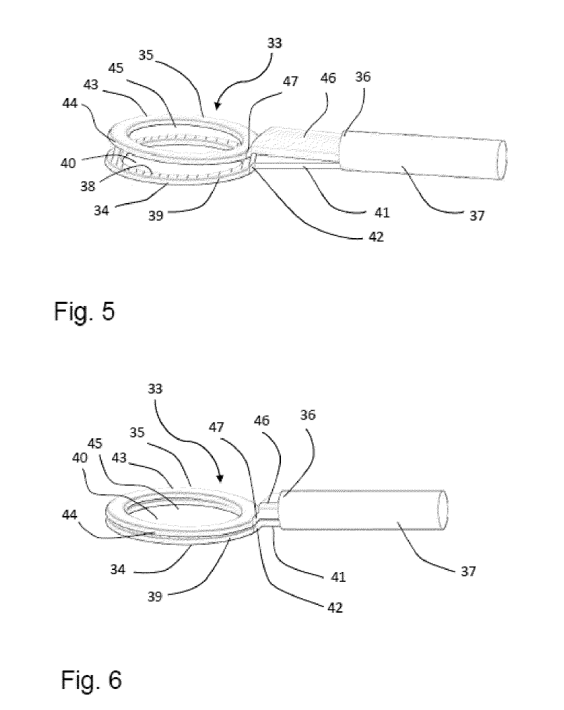

FIG. 5 is an enlarged diagrammatic side view of an end 33 of a preferred embodiment of the microsurgical forceps of the present invention showing the jaws 34 and 35 in an open position and distal end 36 of the tubular shaft 37. The lower jaw 34 is a flat circular ring with anterior surface 38 and rounded edge 39. The anterior surface 38 is the gripping platform and has fine grooves. The lower jaw 34 has a central hole 40. Lower jaw 34 is attached to the lower shank 41 but the lower boundary of the lower jaw is not level with the lower boundary of the shank and is posteriorly offset at the joint 42. The upper jaw 35 is a flat circular ring with anterior surface 43, rounded edge 44 and central hole 45. The posterior surface of upper jaw 35, which is not seen here, is the gripping platform and faces the anterior surface 38 of lower jaw 34. This surface too has fine grooves. Upper jaw 35 is attached to the upper shank 46 but the lower boundary of the upper jaw is not level with the lower boundary of the shank and is posteriorly offset at the joint 47.

FIG. 6 is an enlarged diagrammatic side view of an end 33 of the microsurgical forceps of FIG. 5 showing the jaws 34 and 35 in a closed position and distal end 36 of the tubular shaft 37. The lower and upper jaws 34, 35 are proximate each other in the closed position, such that the anterior surface of lower jaw 34 and posterior surface of upper jaw 35 are apposed to each other. Lower jaw 34 is attached to the lower shank 41 and is posteriorly offset at the joint 42. The upper jaw 35 is a flat circular ring with anterior surface 43 and rounded edge 44 and central hole 45. The central hole 45 is coaxial with the central hole 40 of the lower jaw 34. Upper jaw 35 is attached to the upper shank 46 and is posteriorly offset at the joint 47. The upper shank 46 is thicker and has a greater vertical thickness than the lower shank 41. Distal end 36 of shaft 37 has advanced towards the ring jaws 34 and 35 and now covers most of the length of shanks 41 and 46.

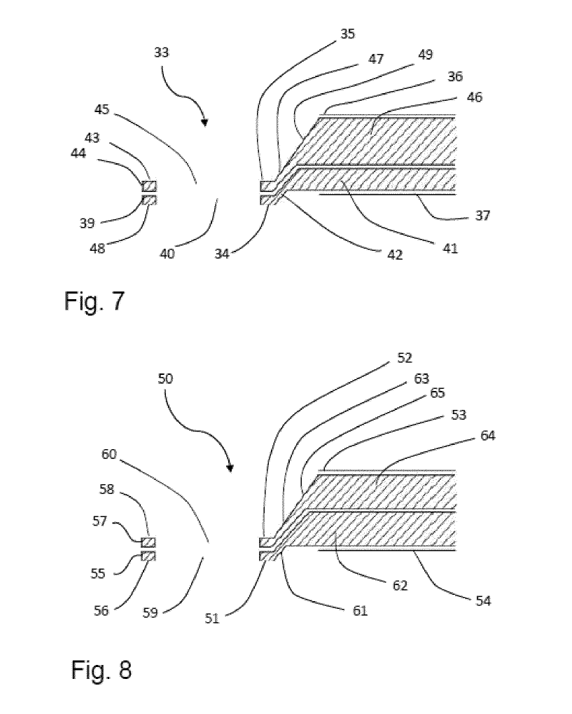

FIG. 7 is an enlarged diagrammatic view of an end 33 of the microsurgical forceps of FIG. 5 showing a longitudinal section through the middle of the closed jaws 34 and 35 and the distal end 36 of the tubular shaft 37 showing sections of posteriorly offset jaws and unequally thick shanks. The section of the lower jaw 34 shows edge 39 and posterior surface 48. The section of the upper jaw 35 shows anterior surface 43 and edge 44. The hole 40 of lower jaw 34 and hole 45 of upper jaw 35 are coaxial. The joint 42 has a beveled posterior surface and posteriorly offsets the lower jaw 34 in relation to lower shank 41. The joint 47 has a beveled anterior surface and posteriorly offsets the upper jaw 35 in relation to upper shank 46. The anterior surface 49 of the shank 46 is beveled where it meets joint 47. The upper shank 46 is vertically thicker than lower shank 44.

FIG. 8 is an enlarged diagrammatic view of an end 50 of an embodiment of the microsurgical forceps of the present invention showing a longitudinal section through the middle of the closed jaws 51 and 52 and the distal end 53 of the tubular shaft 54 showing sections of posteriorly offset jaws and equally thick shanks. The section of the lower jaw 51 shows edge 55 and posterior surface 56. The section of the upper jaw 52 shows edge 57 and anterior surface 58. The hole 59 of lower jaw 51 and hole 60 of upper jaw 52 are coaxial. The joint 61 has a beveled posterior surface and offsets the lower jaw 51 in relation to lower shank 62. The joint 63 has a beveled anterior surface and offsets the upper jaw 52 in relation to upper shank 64. The anterior surface 65 of the shank 64 is beveled where it meets joint 63. The upper shank 64 is vertically the same thickness as the lower shank 62.

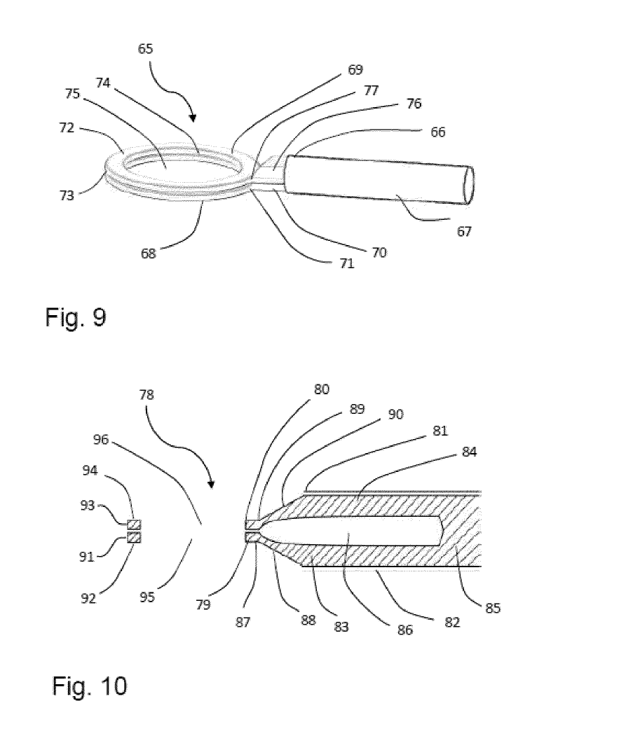

FIG. 9 is an enlarged diagrammatic side view of an end 65 of an embodiment of the microsurgical forceps of the present invention showing the distal end 66 of the tubular shaft 67, the jaws 68 and 69 in closed position with the lower boundary of the lower jaw 68 being coplanar with the lower boundary of lower shank 70. The anterior surface of lower jaw 68 and posterior surface of upper jaw 69 are apposed to each other. The lower boundary of lower jaw 68 is continuous and coplanar with the lower boundary of the lower shank 70 at the joint 71. The upper jaw 69 is a flat circular ring with anterior surface 72 and rounded edge 73 and central hole 74. The central hole 74 is coaxial with the central hole 75 of the lower jaw 68. The lower boundary of the upper jaw 69 is continuous and coplanar with the lower boundary of the upper shank 76 at the joint 77. The upper shank 76 is thicker and has a greater vertical thickness than the lower shank 70. Distal end 66 of shaft 67 covers most of the length of shanks 70 and 76.

FIG. 10 is an enlarged diagrammatic view of an end 78 of an embodiment of the microsurgical forceps of the present invention showing a longitudinal section through the middle of the distal shaft, thin shanks, central rod and closed jaws. The closed jaws 79 and 80 are apposed and are in mid position in relation to the vertical section of the distal end 81 of the tubular shaft 82. The thin shanks 83 and 84 are connected at the central rod 85 to form a U such that there is a gap 86 between them in the closed position of the jaws. Lower jaw 79 is connected to the shank at joint 87. Adjacent the joint 87, the posterior surface 88 of the lower shank 83 is beveled. Upper jaw 80 is connected to the shank at joint 89. Adjacent the joint 89, the anterior surface 90 of the upper shank 84 is beveled. The section of the lower jaw 79 shows edge 91 and posterior surface 92. The section of the upper jaw 80 shows edge 93 and anterior surface 94. The hole 95 of lower jaw 79 and hole 96 of upper jaw 80 are coaxial.

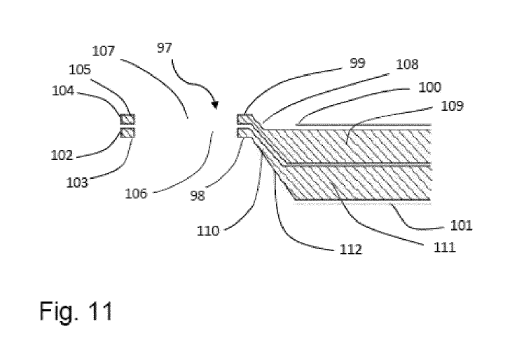

FIG. 11 is an enlarged diagrammatic view of an end 97 of an embodiment of the microsurgical forceps of the present invention showing a longitudinal section through the middle of the closed jaws 98 and 99 and the distal end 100 of the tubular shaft 101 showing sections of anteriorly offset jaws and equally thick shanks. The section of the lower jaw 98 shows edge 102 and posterior surface 103. The section of the upper jaw 99 shows edge 104 and anterior surface 105. The hole 106 of lower jaw 98 and hole 107 of upper jaw 99 are coaxial. The joint 108 has a beveled anterior surface and anteriorly offsets the upper jaw 99 in relation to upper shank 109. The joint 110 has a beveled posterior surface and anteriorly offsets the lower jaw 98 in relation to lower shank 111. The posterior surface 112 of the shank 111 is beveled where it meets joint 110. The lower shank 111 and upper shank 109 are vertically equally thick.

Those ordinarily skilled in the art can make changes in the embodiments described and illustrated, without altering the concepts of the present invention. Hence, it is to be understood that the invention is not limited to the descriptions, illustrations and examples, but includes all modifications within the scope of this invention.

1. A forceps for microsurgery comprising:
a tubular shaft having an outer diameter, a distal end and a proximate end;
a central rod coaxially contained within said tubular shaft, said central rod having a distal end and a proximate end;
an upper shank having a proximate end attached to said distal end of said central rod, and a distal end;
an upper jaw attached to said distal end of said upper shank, said upper jaw having a first diameter that is greater than said outer diameter of said tubular shaft;
a lower shank having a proximate end attached to said distal end of said central rod, adjacent said proximate end of said upper shank, and a distal end; and
a lower jaw attached to said distal end of said lower shank, said lower jaw having a second diameter that is greater than said outer diameter of said tubular shaft;
said forceps having an open position in which said upper jaw and said lower jaw are separated from each other, and a closed position in which said upper jaw and said lower jaw are proximate each other.

2. The forceps of claim 1, wherein said upper jaw includes a first hole, and said lower jaw includes a second hole, wherein said first and second holes are coaxial and cooperate to define a continuous opening when said forceps is in its said closed position.

3. The forceps of claim 1, wherein said upper shank has a first width that is less than said first diameter of said upper jaw, and wherein said lower shank has a second width that is less than said second diameter of said lower jaw.

4. The forceps of claim 1, wherein said upper jaw is attached to said upper shank by an upper joint, and wherein said lower jaw is attached to said lower shank by a lower joint.

5. The forceps of claim 4, wherein said upper joint has a beveled anterior surface, whereby said upper jaw is posteriorly offset from said upper shank, and wherein said lower joint has a beveled posterior surface, whereby said lower jaw is posteriorly offset from said lower shank.

6. The forceps of claim 4, wherein said upper jaw has a posterior surface that is coplanar with a posterior surface of said upper shank when said forceps is in its said closed position, and wherein said lower jaw has a posterior surface that is coplanar with a posterior surface of said lower shank when said forceps is in its said closed position.

7. The forceps of claim 4, wherein said upper jaw has a posterior surface that is coplanar with a posterior surface of said upper shank when said forceps is in its said closed position, and wherein said lower jaw has an anterior surface that is coplanar with an anterior surface of said lower shank when said forceps is in its said closed position.

8. The forceps of claim 4, wherein said distal end of said central rod has an anterior portion attached to said proximate end of said upper shank, and a posterior portion attached to said proximate end of said lower shank, such that said upper shank is separated from said lower shank by a gap therebetween.

9. The forceps of claim 8, wherein said upper shank has a beveled anterior surface adjacent said upper joint, and wherein said lower shank has a beveled posterior surface adjacent said lower joint.

10. The forceps of claim 4, wherein said upper joint has a beveled anterior surface, whereby said upper jaw is anteriorly offset from said upper shank, and wherein said lower joint has a beveled posterior surface, whereby said lower jaw is anteriorly offset from said lower shank.

11. The forceps of claim 10, wherein said lower shank has a beveled posterior surface adjacent said lower joint.

12. The forceps of claim 1, wherein said upper shank has a first thickness, and wherein said lower shank has a second thickness that is less than said first thickness.

13. The forceps of claim 1, wherein said upper shank has a first thickness, and wherein said lower shank has a second thickness that is equal to said first thickness.

14. The forceps of claim 1, wherein said upper jaw has a first thickness and said lower jaw has a second thickness, whereby the combined thickness of said upper and lower jaws facilitates the passage of said first and second jaws through an incision made during ophthalmological surgery.

15. The forceps of claim 1, wherein said first diameter of said upper jaw and said second diameter of said lower jaw are equal.

16. The forceps of claim 1, wherein said second diameter of said lower jaw is greater than said first diameter of said upper jaw.

17. The forceps of claim 1, wherein said upper jaw and said lower jaw have rounded edges.

18. The forceps of claim 1, wherein said upper jaw includes a grooved posterior surface, and wherein said lower jaw includes a grooved anterior surface facing said grooved posterior surface of said upper jaw.

19. The forceps of claim 1, wherein said upper jaw includes a sandblasted posterior surface, and wherein said lower jaw includes a sandblasted anterior surface facing said sandblasted posterior surface of said upper jaw.

20. The forceps of claim 1, wherein said upper jaw includes a diamond-dusted posterior surface, and wherein said lower jaw includes a diamond-dusted anterior surface facing said diamond-dusted posterior surface of said upper jaw.

21. The forceps of claim 1, wherein said upper jaw and said lower jaw are ring-shaped.
